(19) United States
(12) Patent Application Publication
(10) Pub. No.: US 2012/0024979 A1
(43) Pub. Date: Feb. 2, 2012

(54) Air freshener

(76) Inventor: Jeffrey F. Wadlin, Great Falls, VA (US)

(21) Appl. No.: 13/136,268

(22) Filed: Jul. 27, 2011

Related U.S. Application Data
(60) Provisional application No. 61/368,967, filed on Jul. 29, 2010.

(51) Int. Cl. A61L 9/04

(52) U.S. Cl. 239/44; 239/34

(57) ABSTRACT

A diffuser-type air freshener apparatus or appliance (1, 100) affixed to an air grille (6) of a domestic or industrial central air handling system. The appliance includes a housing module which may include a reservoir (24) for storing a volatile fragrant liquid into which one or more scent-wicks (8, 108) are adjustably immersed. Alternatively, dry or pre-moistened wicks may be employed and selectively added or removed to obtain the desired level of scent diffusion. Alternatively, the appliance housing defines an inner opening (42) through which air passes when returning along a defined airflow direction (A) to the central system. One or more flat scent elements or wicks are disposed such that their major flat wicking sides (9) are oriented generally along the defined airflow direction, affording substantial diffusing and/or wicking action with minimal interference with system airflow. Fasteners, including tabs (50) or a detachable clip bracket (29), releasably secure the air freshener appliance to the air grille.

### CROSS-REFERENCE TO RELATED APPLICATIONS

The present invention claims priority from U.S. Provisional Patent Application Ser. No. 61/368,967, filed Jul. 29, 2010, entitled “Air Freshener,” the subject matter of which is incorporated by reference herein.

### BACKGROUND OF THE INVENTION

1. Field of the Invention

The present invention is in the technical field of air fresheners in general, and more particularly, to freshener or scent diffuser units configured for mechanical adaptation to building heating, ventilation and air conditioning (HVAC) systems having air movers.

2. Description of Related Art

While air freshener systems have long existed and many of those have been patented or are subject to patent applications, existing fresheners are known to suffer from one or more effectiveness shortcomings, or pose maintenance challenges to the typical consumer. Conventional diffuser-type air fresheners, such as reed diffusers, are limited to the volume of air into which they can impart a fragrance. This is because widely used fresheners typically are situated on a piece of furniture or the like where ambient air is relatively still, resulting in limited diffusion. Well-known devices also may be limited by the wicking instrument, often comprising natural reeds incapable of wicking and diffusing a volatile fragrant liquid fast enough to create a substantial fragrance concentration in relatively large air volumes typically found in building HVAC air handlers.

Many currently-known and available wicking diffuser air freshener devices are designed for use in central air systems, either internally or attached to an external duct or return air grille. These typically are configured to introduce only a single wick element to be directly engaged by forced airflow. Notoriously, single wick elements present insufficient surface area to impart enough scent to freshen an entire apartment, house, office or building. Still further, known wicking arrangements position solid scent blocks or fluid wicks in a manner focused on significant air flow engagement to maximize scent diffusion while ignoring their obvious degrading impact on airflow efficiency. Finally, existing air freshener devices configured for permanent installation or subject to being repeatedly installed and removed as a single unit, are notoriously difficult to service and maintain. These devices are often ignored, forgotten and eventually left to collect dust and pollen. Moreover, some wicking devices require expensive altering of existing ductwork.

All of the prior art devices to Applicant's knowledge suffer various inadequacies in functional design and operational efficiency, as well as presenting considerable expense and maintenance challenges. The present invention addresses these and other inadequacies of the prior art in a straightforward manner, resulting in an inexpensive air freshener that is exceptionally simple to install, operate and maintain. The proposed configuration provides the requisite surface area to freshen the large volumes of air in an apartment, house, office or building, greatly overcoming the aforementioned problems of the prior art devices and systems. The unique configuration of the instant invention also minimizes airflow disruption. Moreover, it is easily adjustable in terms of scent quantity dispersion, as will be appreciated from the following summary and detailed description.

### SUMMARY OF THE INVENTION

The present invention is a diffuser-type air freshener device preferably designed as a module to be removably fastened to a building's central air system return grille. Indeed, the present invention can find use in HVAC systems in homes, apartments, offices, industrial buildings, theatres, factories, restaurants and so forth. In this context, the phrase modular is defined as a separable or independent unit from its general environment, readily interchangeable with other similar modules and even possessing interchangeable components, the assemblage of which performs its own activity or service. The inventive freshener device includes a removably-mountable housing or frame or a structure defining an airflow opening therethrough for airflow in a generally downstream linear direction from front to rear of the device and into said grille. Within this frame or housing or structure, a unique scent application system is incorporated and designed to be readily adjustable in terms of scent quantity delivered therefrom.

In one embodiment of the present invention, the inventive air freshener module housing or frame optionally incorporates a reservoir chamber for storage of a generally volatile, fragrant liquid. A novel reservoir viewing port facilitates the user's exterior monitoring of wick submersion, liquid level and refilling requirements. This port is in general alignment with the reservoir which is at least in part substantially transparent to enable such monitoring. Further included is at least one generally flat, elongated scent element or member, such as a pre-saturated or presoaked member or a wick, adjustably secured for diffusing the volatile liquid, as a scent, into the circulated air. Each scent diffusion element may be deployed as a single scent member or wick or combined with similarly configured elements. A novel holder arrangement facilitates partial immersion adjustment and/or full removal of at least one scent element relative to the liquid reservoir chamber. This novel adjustability feature facilitates easy moderation or cessation of liquid diffusion and wicking activity.

The present invention includes removable or releasable fasteners for temporarily affixing the inventive air freshener device to a system return air grille. Optionally, at least one pre-moistened wick element can be removably deployed with or without said reservoir chamber, and easily modified for optimum diffusion. Where no reservoir chamber is to be included, pre-moistened or dry wick elements (i.e., deployed singularly or as a plurality) may be deployed as needed and removed or replaced as necessary to maintain a desired scent diffusion effect.

Each scent element or member, e.g., the aforementioned pre-saturated member or wick, is preferably elongated and configured with major, generally flat sides with relatively minor edges. Construction of the inventive air freshener device preferably constrains scent element orientation such that the major sides of each scent element are maintained substantially parallel, or significantly less than perpendicular, to the defined airflow direction (i.e., generally linear across the air freshener device and into its associated return duct grille). Since the major, generally flat sides of each elongated scent element naturally enjoy significantly greater surface area relative to the narrower minor edges, the rate of scent diffusion or wicking is maximized with relatively small, if any, diminution or disruption of air flow through the grille.

Furthermore, in some embodiments, the air freshening device may adjustably support said at least one scent element through means of a friction or interference fit or by pinching or gripping major sides thereof as will be described in detail herebelow. It should be understood that in other embodiments of the invention the scent elements may rely on gravity and be only loosely held in place by combs or like devices. In any case, said at least one scent element or member, such as a wick, fitted into its support is subject to being individually adjusted or removed, such that it is either partially immersed in the fragrant liquid within a reservoir, or entirely out of contact with the fragrant liquid. For example, when at least one wick element is wholly withdrawn from contact with the liquid, e.g., by adjustment or entire removal, the wicking process with respect to the wick eventually ceases as fluid scent dissipates therefrom, thereby disrupting scent delivery through the air handling system. Furthermore, the air freshener module may include a fastener which can be selectively affixed to one or more louvers of an air return grille through means of hooks, clips or equivalent elements. The air freshener device itself is easily detached and replaced to facilitate servicing, such as making wicking adjustments, withdrawing wicks, or for draining, refilling, cleaning and so forth.

### BRIEF DESCRIPTION OF THE DRAWINGS

While the specification concludes with claims particularly pointing out and distinctly claiming the subject matter that is regarded as forming the present invention, it is believed that the invention will be better understood from the following Detailed Description, taken in conjunction with the accompanying Drawings, where like reference numerals designate like structural and other elements, in which:

FIG. 1 is a front perspective view of one embodiment of an air freshener device of the present invention illustrated in a typical operational environment, for example, on a ventilation system return duct grille (shown illustrated in dash-lines);

FIG. 2 is a front perspective view of the air freshener device of FIG. 1;

FIG. 3 is an exploded front perspective view of the air freshener device of FIG. 1, illustrating component parts thereof;

FIG. 4 is a front elevation of the air freshener device embodiment of FIG. 1;

FIG. 5 is a rear elevation of the air freshener device embodiment of FIGS. 1 and 4, showing a freshener fluid supply reservoir chamber positioned therein;

FIG. 6 is a rear elevation of the air freshener unit embodiment of FIGS. 1 and 4 with the freshener fluid supply reservoir chamber removed to reveal the wicking elements;

FIG. 7 is a front perspective view of an alternative embodiment of an air freshener unit pursuant to the present invention with a different housing and connector tab configuration and without a freshener fluid supply reservoir;

FIG. 8 is a front exploded perspective of the alternative embodiment of FIG. 7, depicting wicking elements for insertion therein;

FIG. 9 is a front elevation of the air freshener alternative embodiment of FIG. 7;

FIG. 10 is a rear elevation of the air freshener alternative embodiment of FIG. 7;

FIG. 11 is a side elevation of the air freshener alternative embodiment of FIG. 7;

FIG. 12 is a front perspective view of another embodiment of an air freshener device of the present invention, particularly a configuration to support wick elements therein;

FIG. 13 is a back perspective view of the air freshener device support configuration of FIG. 12;

FIG. 14 is front perspective view of the fully configured and operational air freshener device employing the support configuration of FIG. 12, including the wick elements;

FIG. 15 is a back perspective view of the air freshener device of FIG. 14;

FIG. 16 is a front perspective view of another embodiment of an air freshener device of the present invention in an expanded state;

FIG. 17 is a rear perspective view of the embodiment of the air freshener device shown in FIG. 16 in an expanded state;

FIG. 18 is a front perspective view of the embodiment of the air freshener device of FIGS. 16 and 17 in a collapsed state;

FIG. 19 is a rear perspective view of the embodiment of the air freshener device of FIG. 18 in a collapsed state; and

FIG. 20 is a front perspective view of the embodiment of the air freshener device of FIGS. 16-19 in operation.

### DETAILED DESCRIPTION OF THE INVENTION

The present invention will now be described more fully hereinafter with reference to the accompanying Drawings, in which preferred embodiments of the invention are shown. It is, of course, understood that this invention may, however, be embodied in many different forms and should not be construed as limited to the embodiments set forth herein; rather, these embodiments are provided so that the disclosure will be thorough and complete, and will fully convey the scope of the invention to those skilled in the art. It is, therefore, to be understood that other embodiments can be utilized and structural changes can be made without departing from the scope of the present invention.

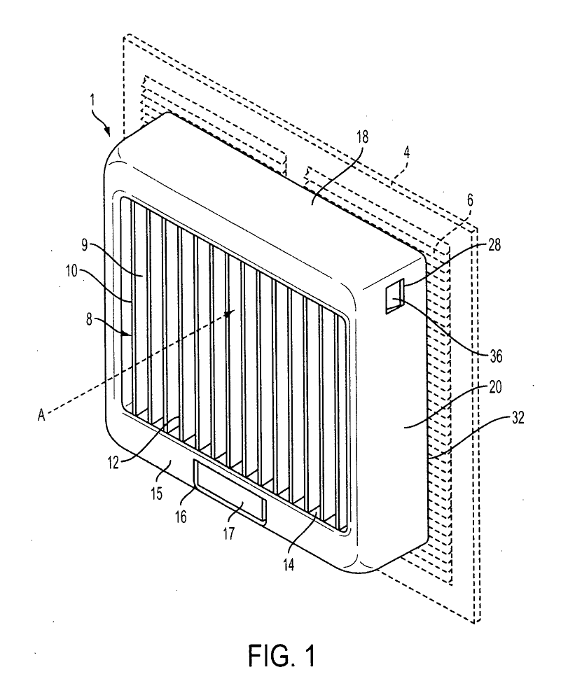

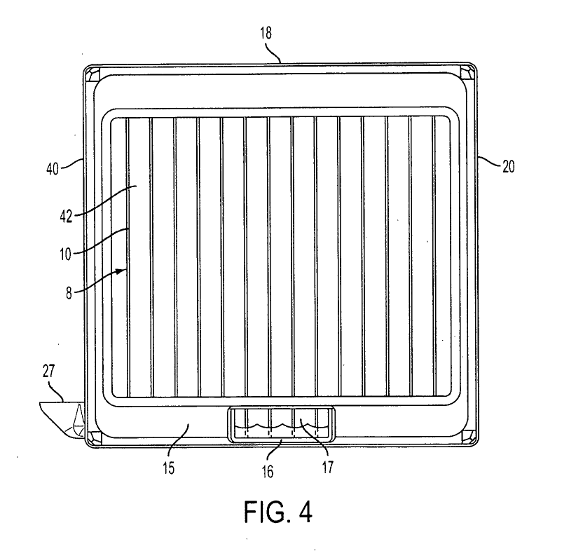

Referring now to the present invention in more detail, FIG. 1 illustrates a first embodiment of the air freshener appliance, device or module of the present invention, designated by the reference numeral 1, removably affixed in place for operation (in a manner to be shown herebelow), such as a central air system return 4, more specifically to return grille 6. The air freshener device 1 includes a housing defined as including the general form of a frame with a top 18 and side extent 20 which, along with correspondingly opposing frame sides, 15 and 40 (as shown in FIG. 4) respectively, define at their inside surfaces an airflow opening (42 shown in FIG. 4) therethrough, extending in a generally linear direction A from a front frame input side to a rear frame output side where said housing engages return grille 6 at device 1/grille 6 interface or rear surface thereof, generally designated by the reference numeral 32. It should, of course, be understood that the airflow may alternatively be outward, i.e., in a direction opposite A.

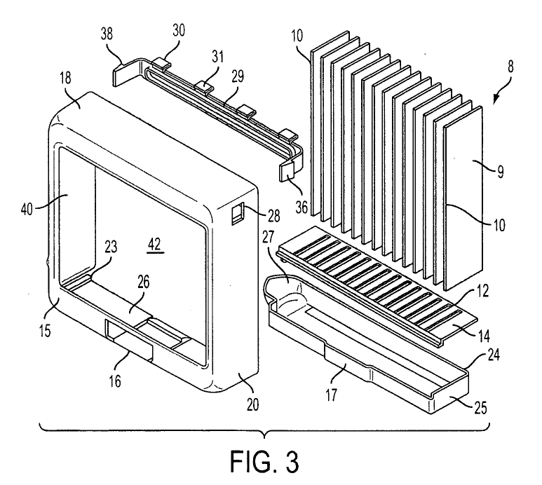

Partially viewable in FIG. 1 is at least one generally flat, elongated scent element or member 8, such as wicks, though not intended as limiting in terms of wicks or the number thereof, which can be one or more. It will be apparent that scent elements 8 include major, generally flat sides 9 and relatively minor or narrower leading and trailing edges 10. Said scent elements 8, which can also include pre-saturated or presoaked members, are adjustably fitted in their respective positions within slots or lid openings 12 in lid 14 of a scent reservoir or reservoir chamber 24 (to be described with respect to FIG. 3). As will be understood, the orientation of slots or lid openings 12 with respect to the above-noted airflow is in the aforementioned generally linear direction A for efficient operation of the central air system.

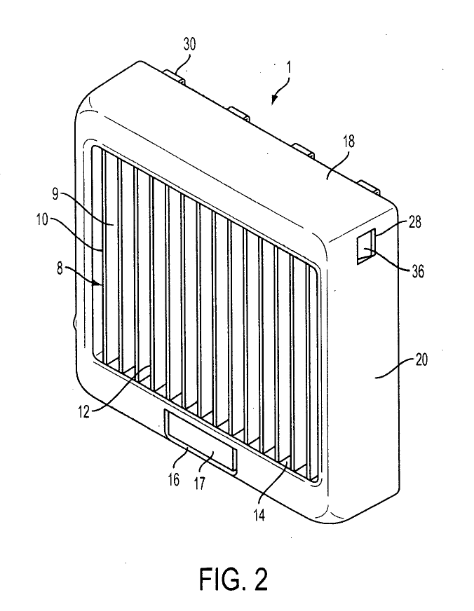

Also seen in FIG. 1 of this first embodiment is a viewing opening or port 16 to reservoir viewing window 17 which itself is a part of a reservoir chamber 24 residing within air freshener device 1, and fully explained with reference to FIG. 2. Viewable through a second opening 28 in the air freshener 1 device housing is clip bracket detent 36 (to be referred to as “right detent 36”), as viewed in FIGS. 1-3—again, fully explained in reference to other drawing figures, as follows.

Turning to FIG. 2, connector or fastener clips 30 (four are illustrated, but one or more may suffice) are seen along a rear edge of housing frame upper extent 18, and are for mounting the air freshener appliance 1 in place for operation, typically on a return vent grille. The view of clips 30 is, of course, obstructed in FIG. 1 by one or more grille louvers 6. Clips 30 are depicted more clearly in FIG. 3 as one or more spring clip wedges affixed to, or integral with, a clip bracket 29. Each clip 30 is furnished with at least one clip surface ridge 31 to ensure frictional securement when inserted between grille 4 louvers 6. Clip bracket 29 further carries a set of detents, namely right detent 36 and left detent 38, configured to snap into air freshener 1 housing frame side extents 20 and 40 (FIG. 4) respectively, as is understood in the art. With this novel bracket connector arrangement, the air freshener appliance 1 may be removed from the bracket 29 for servicing and so forth without disturbing the connector engagement to a vent grille. Bracket 29 not only secures clips 30 to air freshener 1 housing, but also secures other parts (now to be described) of the air freshener 1 unit in place.

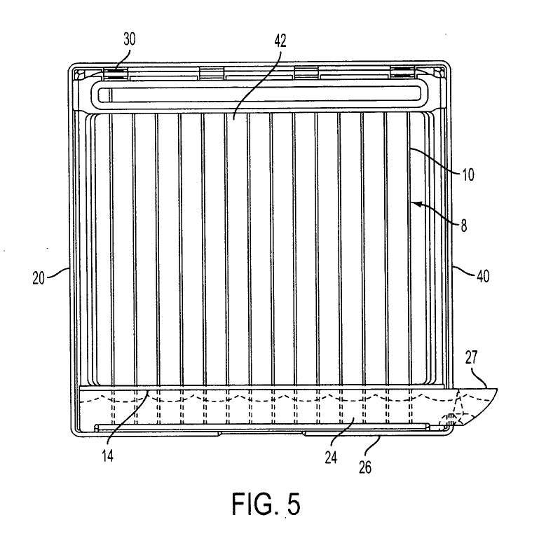

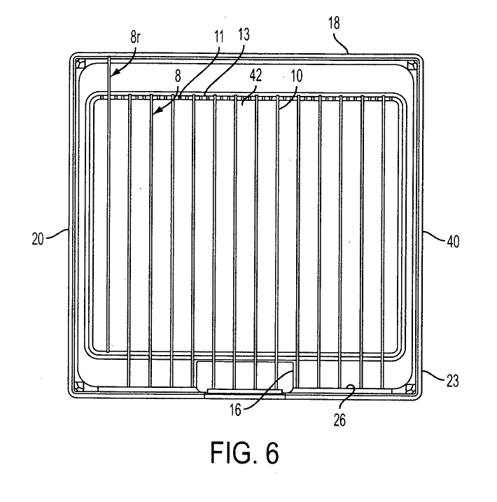

As best seen in the exploded perspective FIG. 3, the air freshener 1 housing, having frame sides 18, 20, 15 and 40 (defining therein airflow opening 42), includes the reservoir chamber 24 at frame bottom 15 on which rests reservoir chamber 24, supported by a reservoir chamber support surface 26 (see FIGS. 5 and 6). The chamber 24 is to be charged with volatile scent fluid and, as shown in FIG. 5, inserted from the aforementioned rear surface 32 of air freshener 1, such that a chamber filler spout extends outwardly through opening 23 in frame side extent 40 (see FIG. 4). Reservoir chamber 24 includes a sidewall 25 which is, preferably, at least in part, substantially transparent as depicted. At least, the aforedescribed reservoir chamber view window 17 (a portion of sidewall 25) should be substantially transparent. This arrangement is best depicted in (assembled) FIG. 4. Convenient viewing port 16 is in general visual alignment with the view window 17, which is substantially transparent part of reservoir chamber wall 24. Again, all of reservoir chamber 24 can be transparent, if so desired. The sidewall 25 is, at least in part, formed as the view window 17, such that a volatile fluid level therein is viewable by a user via viewing port 16 when the reservoir chamber 24 is properly positioned for operation (note FIGS. 4 and 5). This feature enables the user to check the volatile fluid level as well as the extent of wick element immersion, and to do so without dismantling, disconnecting or otherwise removing the air freshener 1 appliance from its station.

Lid 14 is configured to enclose an upper extent of reservoir chamber 24 along a top edge thereof and the sidewall 25, except of course for the chamber filler spout 27, which is configured to protrude outwardly from the housing 1 when fully assembled. Further illustrated in exploded perspective FIG. 3 is a set of scent elements 8 depicted as juxtaposed to lid 14 with lid slots or holder openings 12 specially configured to retain respective ones of said scent elements 8 therein. When assembled, scent elements 8, such as the aforementioned wicks, extend downwardly through the aforementioned lid slots or holder openings 12 into the reservoir chamber 24 to an extent affording partial submersion into volatile fragrant liquid located therein. Said scent elements 8, of course, extend upwardly therefrom so as to be subjected to moving air through the air freshener device or apparatus 1 housing, as described hereinabove.

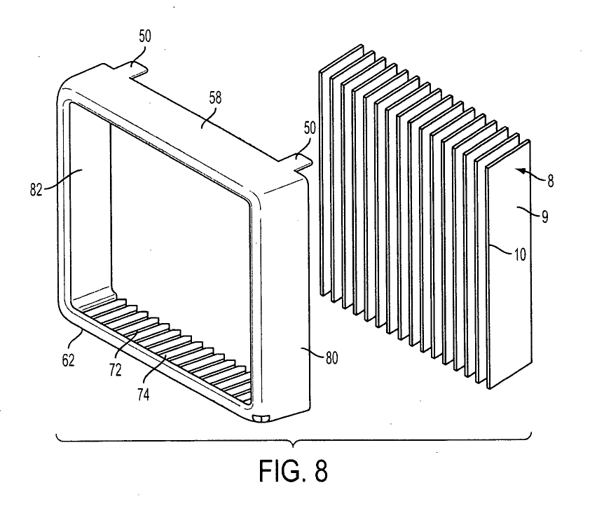

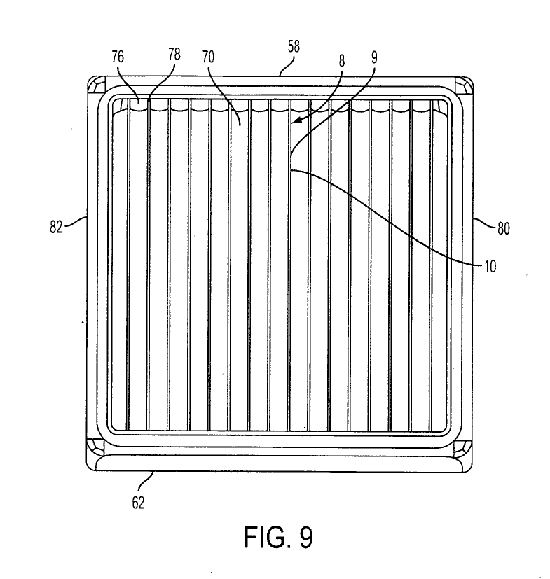

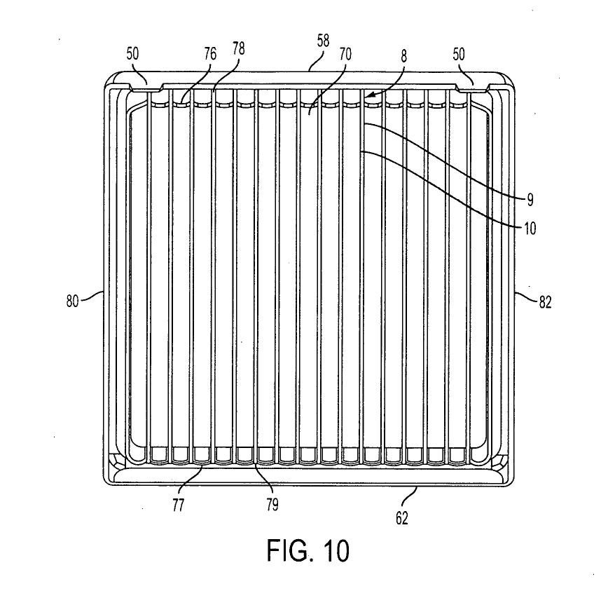

With reference now to FIG. 6 of the Drawings, a plurality of slightly flexible finger elements 11 cooperate to define slots 13 therebetween (generally similar to the fingers/slots 74/72 in FIG. 8, the fingers/slots 76/78 in FIG. 9, and the fingers/slots 76/78 and 77/79 respectively, in FIG. 10, as discussed hereinbelow). These fingers 11 optionally could be mutually divergent, wherein said slots 13 become progressively narrower in width so as to facilitate scent element 8 insertion/removal. In any case, it should be understood that the fingers 11 can take a variety of forms, and preferably cooperate to establish a narrow slot therebetween, such that a scent element can be inserted therein and may, for added stability, be pressed-in to achieve a friction fit. For simplicity, the term “fitted” is herein applied to all such finger/slot interaction with scent elements. For example, an upper extent of each scent elements 8 may be adjustably and releasably friction-fitted or wedged into a defined slot through a slight pinching action or interference fit between the cooperating fingers. With only slight pressure, the scent elements 8 can be individually or collectively raised (e.g., note wicking element 8 in FIG. 10) so as to be immersed to a lesser extent, and sufficiently raised above the volatile fluid surface so as to control liquid diffusion, wicking absorption or capillary activity, as is apparent to those of skill in the art. It is, therefore preferred that sufficient clearance space be afforded within an upper area of the air freshener 1 housing to accommodate upward adjustment of scent elements 8, as needed. Adjustment of scent elements 8 is preferably achieved without otherwise disturbing or removing the air freshener device 1 from its operative station.

It is important to note that: (a) the number of scent elements 8 illustrated in FIG. 3 (and elsewhere) is merely exemplary and shall in no way be considered limiting with respect to the scope of the present invention (as set forth in the appended claims), which can employ one or more such scent elements or wicks; (b) wicking elements that partially immerse in the reservoir 24 are preferred in this embodiment employing scent liquids or fragrances for wicking action; of course, combinations of wicks and other scent members 8 are possible as well; (c) the lid slots or holder openings 12 and finger elements/slot holders 11/13 (as well as the aforementioned fingers/slots 72/74, 76/78, 77, 79) are purposely directionally-oriented so as to constrain the scent elements 8 in substantial alignment with, or substantially parallel to, the above-noted airflow generally in linear direction A (FIG. 1). This approach maximizes the exposed surface area, affording maximum diffusing and wicking activity with minimum airflow disruption.

Reservoir chamber 24 may be easily removed (following detachment of the air freshener device 1 from its grille-mounted position) and replenished, as may be desired. Further, the reservoir chamber 24 may be removed and pre-moistened (or dry) or pre-saturated elements 8 deployed in appropriate number to achieve optimal or desired scenting results. Adjustments in the latter case are made by removing the air freshener device 1, adding or subtracting scent elements 8 and replacing the device 1. An alternative embodiment of the present invention, employing a simpler construction configured specifically for the dry or pre-moistened or pre-saturated element 8, will now be addressed.

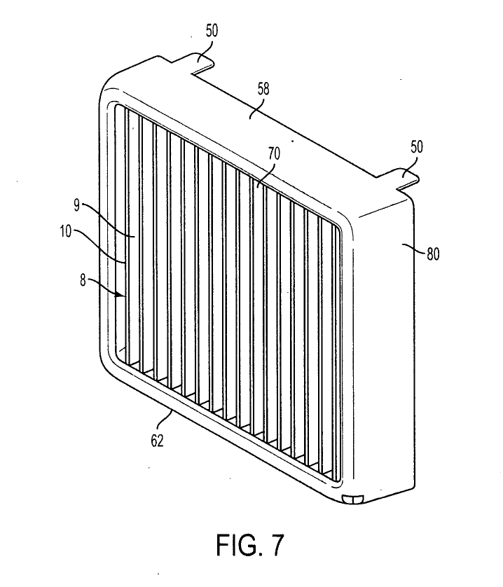

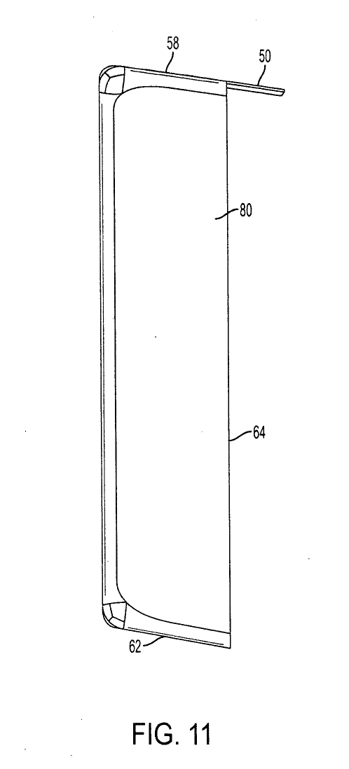

With reference now to FIGS. 7-11 of the DRAWINGS, there is illustrated therein an alternative embodiment of the present invention, which comprises an air freshener device 1 housing similar to the earlier-described version in that it includes a housing defined as taking the general form of a frame with an upper extent or wall 58 and side extent or wall 80 which, along with correspondingly opposing frame extents/walls, 62 and 82 (FIGS. 9, 11) respectively, define an airflow opening (70 in FIG. 10) therethrough, extending in a generally linear direction A (same as in FIG. 1) from a front frame input side to a rear frame output side where said housing engages its associated return grille at device/grille interface 6 of the central air system return 4, as described in connection with the first embodiment.

Viewable in FIGS. 7, 8 and 9 is at least one substantially flat elongated scent element 8, with its two major, generally flat sides 9 and two minor (narrow) edges 10, as afore-described. These scent elements or members 8, however, are dry or pre-moistened or pre-saturated with a substantial charge of volatile fragrant fluid. Such pre-moistened scent elements 8 typically are supplied and stored in sealed packets or other air-tight containers. In this second embodiment, the air freshener 1 device is charged or supplied with scent elements 8 numbering one or more depending upon the desired fragrance diffusion activity or potency. Again, the securement and orientation of the scent elements is important, as now will be discussed.

As viewed near the inside of top wall 58 (see FIG. 10), element holding fingers 76 defining therebetween element holding slots 78 serve both to secure scent elements 8 and directionally orient them in substantial alignment with, or substantially parallel to, the above-noted airflow in generally linear (downstream) direction A (see FIG. 1) common to both embodiments herein described. Again, this orientation of the generally flat scent elements 8 affords maximum diffusing or wicking activity with minimal interference with air flow or degradation of airflow efficiency. For stable performance, a second set of fingers/slots 77/79 is provided adjacent to the air freshener 1 housing bottom wall 62. These lower scent element holders are operable in the same manner as fingers/slots 74/72 (FIG. 8) though having slightly different profiles, but oriented in the same general direction and for the purpose discussed above. It should be understood that all finger/slot profiles could be essentially identical or differing considerably, as long as they structurally cooperate to define a wick-holder slot therebetween. Auxiliary storage space is defined between the wick holders and interior walls of said air freshener housing for holding a reserve supply of wicking elements 8. For example, as shown in FIG. 6, a reserve scent element 8r, along with any other such elements, may be stored in a side portion of the device 1, away from the fluid in the reservoir chamber 24, and preferably sealed to prevent fragrant dispersion due to proximity to the air flow.

Since this second embodiment obviously is inherently lighter in weight than the first embodiment, described as including a reservoir chamber 24 with fluid supply, a simpler grille louver attachment is contemplated in planar tabs 50, illustrated in FIGS. 7, 8, 10 and 11. One or more such tabs (or equivalent hooks, clips, and so forth) may be appropriately positioned along a rear face 64 of air freshener 1 housing, as an integral or add-on rearward extension of air freshener 1 housing frame upper extent 58. To facilitate interconnection of the air freshener device and return vent, the housing frame upper extent 58, along with its associated planar tabs 50, are sloped slightly inwardly of the frame toward the defined airflow linear direction. The angular insertion of the planar tab into a vent louver 6 is thereby simple and more secure. This latter embodiment could be produced as a disposable modular unit with integrated wicking elements to be wholly discarded or recycled when depleted.

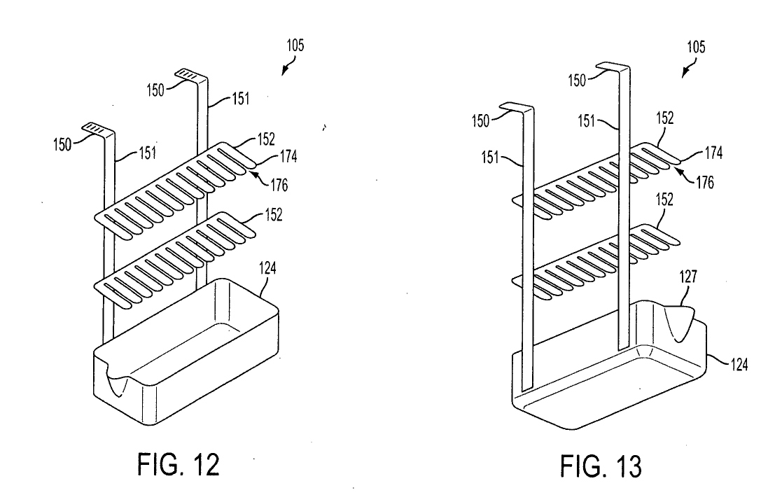

It should be understood that the aforementioned configurations of the air freshener components illustrate some preferred embodiments of the present invention. A further alternative configuration is set forth in FIGS. 12-15.

With reference now to FIGS. 12 and 13, there is shown an alternative configuration of the air freshener device of the instant invention, particularly a support member, generally designated by the reference numeral 105, which can be removably affixed to the aforementioned central air system return 4, such as at grille 6 (as shown in FIG. 1). The support member 105 is secured to the aforedescribed grille 6 via planar tabs 150, which enter therein and engage, e.g., by action of gravity and/or friction, as described. As shown in FIG. 12, the planar tabs 150 are the top portion of vertical supports 151, which are at right angles thereto and disposed downward. Attached to the vertical supports 151 are two or more combs or scent element holders 152.

As shown in FIGS. 12 and 13, each element holder 152 has a number of spaced element holding fingers 174 extending horizontally therefrom, defining therebetween element holding slots 176 to both secure respective scent elements 108 (shown in FIGS. 1.4 and 15) therein and directionally orient them to the air flow A (shown in FIG. 1), as described hereinabove in connection with the prior embodiments. Fragrant or scented fluid may be stored within a reservoir chamber 124, which is also attached to the vertical supports 151, as shown in FIG. 13, and could also serve as a catch of any excess fragrant or liquid, e.g., dripping off of presoaked, pre-moistened or pre-saturated scent members or overly-saturated wicking elements 8. Additionally, as with the prior embodiments, lower scent element holders may be employed within the reservoir 124 to further orient the various scent elements 108. A spout 127 can be used for pouring the contained fluid, as described.

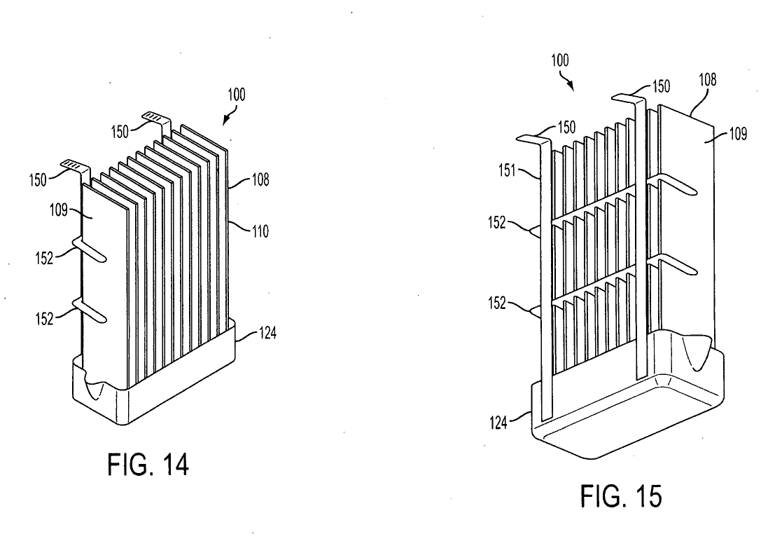

With reference to FIGS. 14 and 15, there is shown a fully operational embodiment of the air freshener of the present invention using the support framework described in FIGS. 12 and 13, designated generally by the reference numeral 100, which in operation is placed over a grille louver 6, as herein described (and illustrated in FIG. 1). The evenly-spaced, vertically-oriented scent members or wicks 108, interlaced in respective fingers 174 and slots 176 of the element holders 152, provide enhanced surface area exposure to the air flow A, as discussed.

The even further simplified design of this embodiment makes the configuration lighter in weight easier for the consumer to use than the earlier embodiments. Although not as robust as the aforedescribed four-walled configurations, this design offers many advantages over the framed embodiments, particularly in having fewer constituent parts, minimizing assembly, maintenance and complexity.

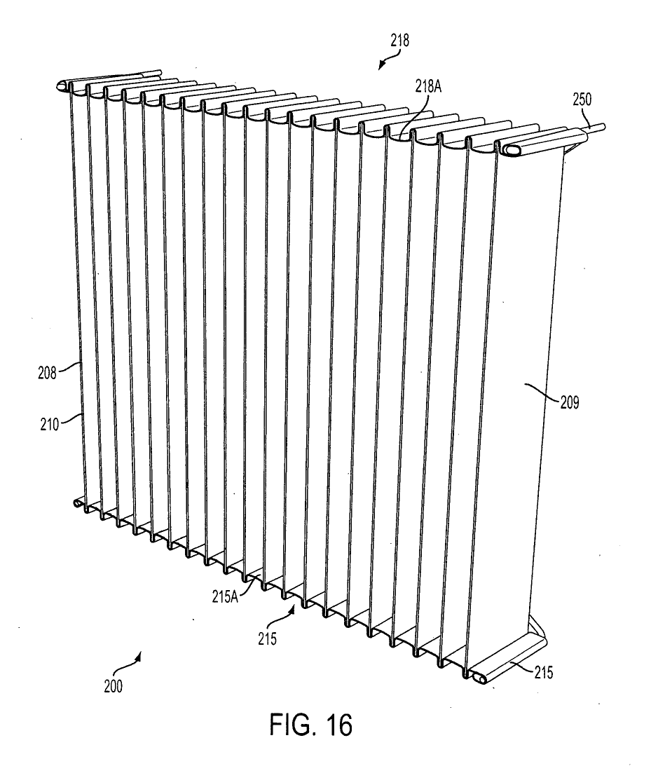

It should be understood that the configurations of the air freshener components described above illustrate some preferred embodiments of the present invention. Another alternative configuration is set forth in FIGS. 16-20, which is even simpler and lighter in weight than the previous embodiments.

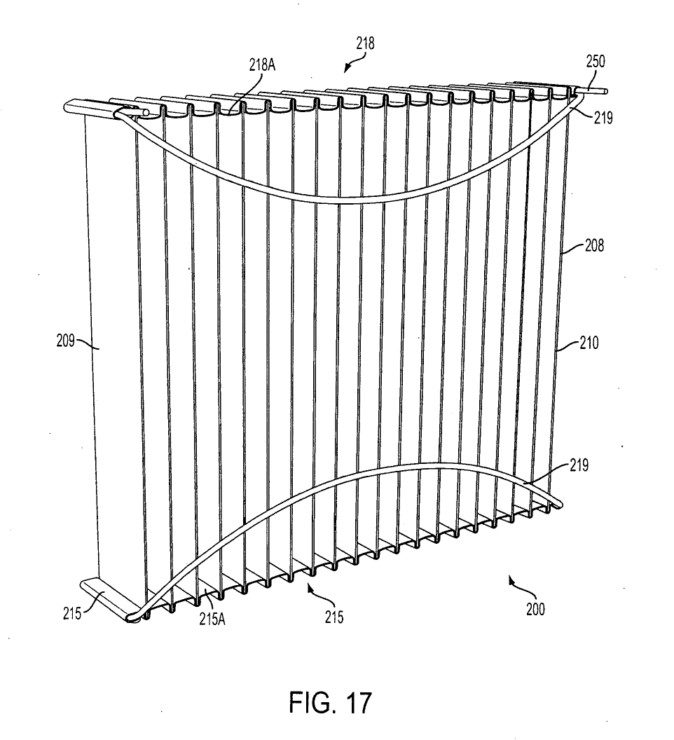

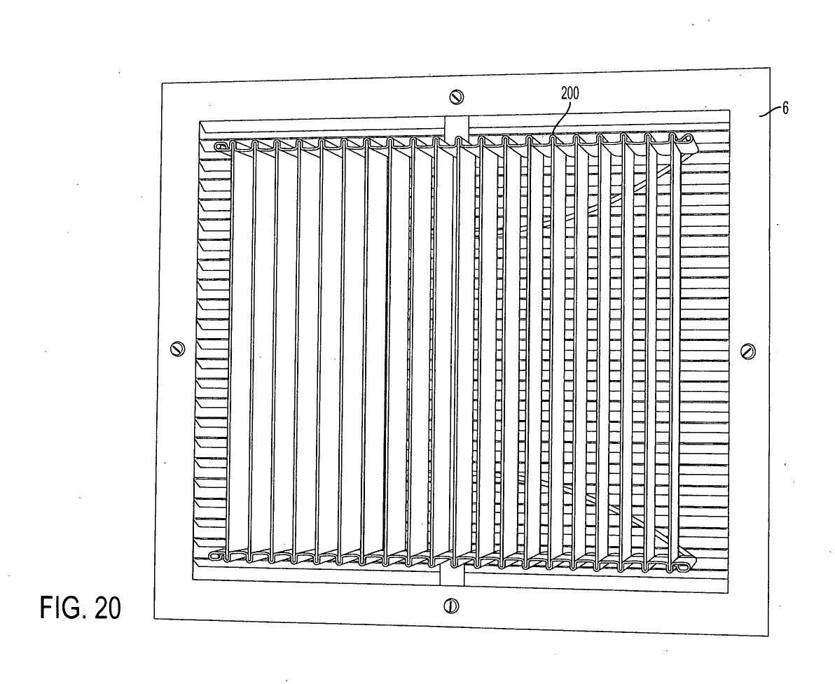

With reference now to FIG. 16, there is shown a front view of a collapsible air freshener device, generally designated by the reference numeral 200 in a fully extended state. As is readily apparent, this configuration does not employ a reservoir or other such supply of liquid fragrance or scent. Instead, the device, more particularly the scent elements are preferably pre-dipped and come out pre-scented, pre-moistened, pre-saturated or pretreated for scent, which diffuses from the device 200 upon exposure to air. A rear view of the device 200 is shown in FIG. 17. This embodiment, as with the previous embodiments, has a number of scent elements or members 208, e.g., the aforementioned at least one substantially flat elongated scent element, with its two major, generally flat sides 209 and two minor (narrow) edges 210, as described. A pair of tabs 250, described further hereinbelow, extend from the back of the device 200 to engage the aforementioned grille louver 6, as herein described (and illustrated in FIGS. 1 and 20).

It should be understood that the air freshener device 200 in this embodiment is preferably a disposable unitary construction. Instead of fixed fingers and slots, as in the prior embodiments, the scent elements 208 are flexibly secured to upper and lower flexible connectors, generally designated by the reference numerals 218 and 215, respectively. As shown in FIGS. 16 and 17, the connectors 215 and 218 have respective flexible members or spacers 215A and 218A, such as fabric, between the respective scent elements 208, defining therebetween gaps and directionally orienting the scent members 208 in substantial alignment with, or substantially parallel to, the above-noted airflow in generally linear (downstream) direction A (see FIG. 1) common to the embodiments herein described.

As shown in FIG. 17, a pair of hanging loops, wires or springs 219 provide support for the device 200 in operation. For example, the wires 219 in FIG. 17 are shown in an expanded state, pushing the device 200 open, and stretching the connectors 215 and 218 outward, thereby opening the aforementioned air passage therethrough, as described. As shown, the wires 219 have end portions that are bent to protrude rearward for engaging the HVAC intake grille louver 6, as described. In other words, these end portions of the wires form the tabs 250.

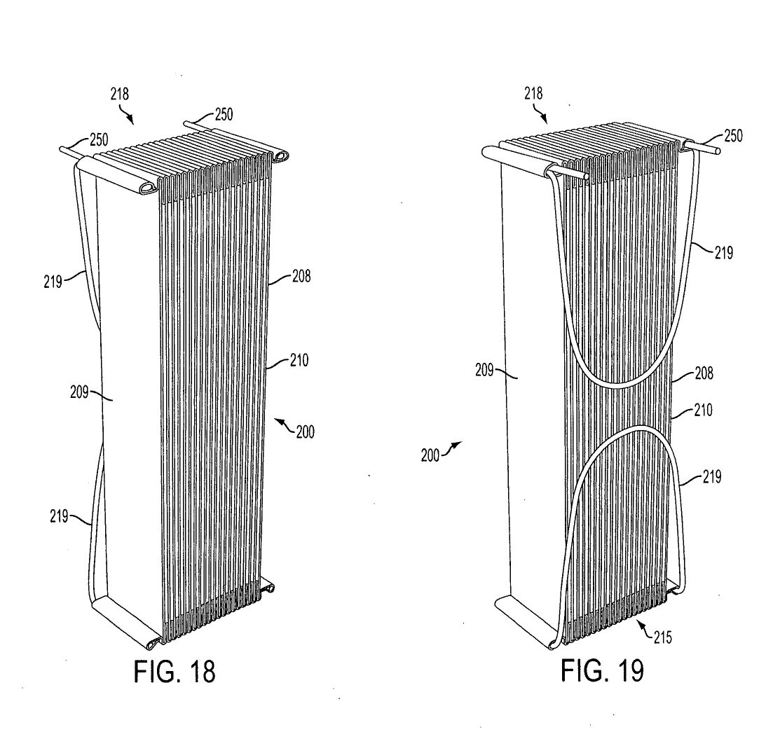

With reference to FIGS. 18 and 19, there are illustrated the air freshener device 200 of FIGS. 16 and 17 in a collapsed state. As shown, the wires 219 are in a compressed state, and the scent members 208 are collapsed against each other and compressed. The respective connectors 215 and 218 therebetween are also collapsed. The device 200 is preferably shipped in this compressed state in a sealed bag or box to preserve the fragrance or scent therein until use. It should also be understood that the device 200 may be configured for re-saturation or re-soaking for reuse, thereby minimizing waste and helping the environment.

With reference now to FIG. 20 of the DRAWINGS, there is illustrated the usage of the air freshener device 200 over the aforementioned grille louver 6. As illustrated, the expanded device 200 may substantially cover the grille 6, and by virtue of the spacing of the respective scent elements 208 and the spring action of the wire 219 the air passing therethrough is minimally affected. After a period of time, the scent or fragrance in the air freshener 200 will diminish, and the entire device 200 may be easily removed, discarded and replaced. Alternatively, an alternate fragrance or scent may be desired and the device 200 removed and stored in a sealed bag or box to maintain the scent therein for later use.

Although the wires 219 in this embodiment expand outward and are not adjustable, it should be understood that the device 200 in an alternate configuration is adjustable over the vents, e.g., the device 200 may omit the wires 219 (except for the tabs 250), permitting loose adjustability over the vents, limited only by the extent of the flexible materials 215A and 218A.

It should further be understood that other configurations, not expressly set forth herein, are considered within the purview of the present invention, particularly as set forth in the claims hereinbelow.

The novel air freshener module described herein in its various embodiments may be made of plastic or other sufficiently durable and fluid-tight material, such as wood, ceramic, metal, and the like. Plastic parts, of course, can be inexpensively fabricated and shipped, and may be easily cleaned as necessary or discarded altogether. Moreover, every part of the presently-described invention can be fabricated entirely of dishwasher safe plastic. In fact, the entire air freshener module, except for the scent elements, can be detached from its grille and placed as is, or disassembled, into a household dishwasher for thorough cleaning. The reservoir chamber and its associated lid may be transparent, translucent or opaque, with exception of the reservoir viewing window which must be sufficiently transparent to permit an observer to determine a fluid level. Further, the fragrant liquid deployed in the diffusing/wicking action may comprise an oil, alcohol, water or some other suitable base or mixture.

The scent elements 8/108/208 discussed herein may be made of a plastic material with diffusing/wicking properties, or other suitable diffusing and/or wicking material, such as natural or man-made fibers or cloth, plants such as reeds, and the like, or a combination of such materials, as is understood in the art. It should, therefore, be understood that the terms scent elements, scent members or wicks used herein are employed to exemplify types of diffusing materials, wicking materials or both. The usage of a particular configuration, e.g., with a reservoir 24, will determine the types of materials, as is understood in the art.

The fastener clips may be fabricated or formed of plastic, metal or other sufficiently rigid and strong material or combinations of materials, such as plastic, wood, and the like. Again, the air freshener devices described herein may employ multiple or single fasteners, depending on desired stability. Fasteners associated with the air freshener housing frame may be secured to the upper rear of the frame as shown, or can be attached appropriately to housing sides or other location as may be desired.

The present invention offers a number of significant advantages. The novel air freshener module allows a fragrance to be distributed by airflow throughout an entire house, apartment, office or other building or facility structure. The module may be secured to the outside of a return air grille louver so that it is easily accessible by the user, as opposed to requiring the user to access complex inner workings of their HVAC system, such as metal ductwork, bonnet or air filter housings. It should, of course, be understood that the module may be placed on air intake as well as output vents, depending on the dynamics of the system. The air freshener module may be recharged in situ or readily detached for charging, cleaning or servicing. The fluid and/or scent elements can be easily replaced, allowing the user to introduce different fragrances as desired at different times, or to replenish a single fragrance. The air freshener device is alternatively operational with pre-moistened or pre-saturated scent members instead of filling the fluid reservoir, or initial unit construction may omit the reservoir chamber so as to operate only with pre-moistened diffusers.

Moreover, the inventive module device can easily be removed from its fastened position on a vent grille to terminate diffusion when a user does not wish to have fragrance distributed in the airflow. A plurality of scent elements can be deployed to provide sufficient cumulative surface areas for such diffusion. Alternatively, a single scent element can be deployed for minimal diffusion as desired. To ensure quality airflow through the air handler, each scent element is positioned such that its major surface are in substantial alignment with, or substantially parallel to, a defined airflow direction (through the device and into the vent) so as not to impede or disrupt efficient airflow. This arrangement provides optimum diffusion and wicking action with minimal interference with HVAC circulation, as described in more detail hereinabove.

A kit of the components of the instant invention includes the framework or support mechanisms, respective scent elements 8/108/208, and ways to insert and position the scent elements into an air flow, such as on an HVAC or other grille 6. The kit also includes instructions on the usage of the components, e.g., the option to not employ the reservoir chamber 24, such as when using pre-moistened scent elements. The kit also includes instructions on the configurations of the scent elements, e.g., although the examples illustrate the usage of vertically-oriented scent elements, other configurations are, of course, possible, including horizontal disposition and angular. So long as the scent elements so configured due not overly interfere in the operation of the air vent, almost any scent element orientation is possible. Packages of the scent elements, e.g., containing differing fragrances, can be sold separately, preferably sealed if pre-moistened. Of course, blank scent elements may also be sold in such packages, whether or not sealed. The entire device, such as device 200, can come assembled, albeit in a collapsed state, for shipment and fully deployed at use. The devices in the embodiments herein may be susceptible to reuse or discarded.

While the foregoing written description of the invention enables one of ordinary skill to make and use what is considered presently to be the best mode thereof, it should be understood that various changes, substitutions and alterations can be made herein without departing from the spirit and scope of the invention as defined by the appended claims. Therefore, the invention is not to be limited by the above described embodiment, method, and examples, but by all embodiments and methods within the scope and spirit of the invention as claimed.

I claim:

1. An air freshener apparatus comprising:
a support, said support having attachment means to attach said air freshener apparatus to a vent having air flow therethrough; and
a plurality of scent members attached to said support, said scent members having scent surfaces disposed substantially parallel to said air flow,
whereby, through air flow across the scent members, scent in the scent surfaces is transferred to the air flow.

2. The air freshener apparatus according to claim 1, wherein said attachment means attaches said air freshener apparatus to a vent selected from the group consisting of an air intake vent and an air output vent.

3. The air freshener apparatus according to claim 1, wherein said attachment means is selected from the group consisting of a mounting bracket, removable mounting bracket, tabs, detachable clip brackets, and clip brackets.

4. The air freshener apparatus according to claim 1, further comprising:
alignment means for aligning said plurality of scent members along said vent substantially parallel to said air flow.

5. The air freshener apparatus according to claim 4, wherein said alignment means is selected from the group consisting of slots, spacers, fingers, combs and combinations thereof.

6. The air freshener apparatus according to claim 1, further comprising:
at least one comb mechanism, said at least one comb mechanism attached to said support and having a number of fingers and slots for adjustably receiving one or more of said plurality of scent members therein, said comb mechanism supporting and orienting said scent members to said air flow.

7. The air freshener apparatus according to claim 1, wherein said scent surfaces of said plurality of scent members are substantially rectangular, having two major side surfaces and thin edge surfaces, said major side surfaces substantially parallel to said airflow and said thin edge surfaces substantially facing and without substantially impeding said air flow.

8. The air freshener apparatus according to claim 1, wherein said plurality of scent members are pre-treated to contain scent.

9. The air freshener apparatus according to claim 1, wherein said plurality of scent members are composed of material selected from the group consisting of plastic, natural fiber, man-made fiber, cloth, plants and combinations thereof.

10. The air freshener apparatus according to claim 1, wherein said support comprises a housing structure, sides of said housing structure defining an air flow pathway therethrough, and said plurality of scent members disposed therein.

11. The air freshener apparatus according to claim 1, wherein said support, and said plurality of scent members are modular components.

12. The air freshener apparatus according to claim 11, wherein said modular components are dishwasher safe.

13. The air freshener apparatus according to claim 11, wherein said plurality of scent members are disposable.

14. The air freshener apparatus according to claim 1, wherein said support, and said plurality of scent members are a unitary construction.

15. The air freshener apparatus according to claim 1, wherein said air freshener apparatus is disposable.

16. The air freshener apparatus according to claim 1, wherein said air freshener apparatus is collapsible.

17. The air freshener apparatus according to claim 16, further comprising:
collapsing means for collapsably connecting said plurality of scent members to said attachment means.

18. The air freshener apparatus according to claim 16, further comprising:
spring means for expanding a said plurality of scent members apart for deployment.

19. An air freshener apparatus comprising:
a support, said support having attachment means to attach said air freshener apparatus to a vent having air flow therethrough;
a scent reservoir, said scent reservoir attached to said support; and
a plurality of scent members attached to said support, said scent members having an end thereof disposed in said scent reservoir, and having scent surfaces disposed substantially parallel to said air flow,
whereby, through wicking in the scent members, fluid in the scent reservoir transfers scent to the air flow.

20. The air freshener apparatus according to claim 19, wherein said scent reservoir stores a volatile fragrant liquid, said plurality of scent members being adjustably disposed therein for wicking said liquid.

21. The air freshener apparatus according to claim 19, wherein at least one scent member is not disposed in said scent reservoir.

22. The air freshener apparatus according to claim 19, further comprising:
at least one window means for observing fluid in said scent reservoir.

23. The air freshener apparatus according to claim 19, wherein said support, said scent reservoir, and said plurality of scent members are modular components.

24. The air freshener apparatus according to claim 23, wherein said modular components are dishwasher safe.

25. The air freshener apparatus according to claim 19, wherein said plurality of scent members are disposable.

26. A kit for an air freshener device comprising:
a support, said support having attachment means to attach said support to a vent having air flow therethrough;
a plurality of scent members attached to said support, said scent members having scent surfaces disposed substantially parallel to said air flow; and
instructions on assembling said air freshener device.

27. The kit according to claim 26, further comprising:
a scent reservoir attached to said support, at least one of said scent members attached to said support having an end thereof disposed in said scent reservoir,
whereby, through wicking in the scent members, fluid in the scent reservoir transfers scent to the air flow.

28. The kit according to claim 27, further comprising:
a container of fragrant fluid for said scent reservoir.

29. The kit according to claim 26, wherein said plurality of scent members in said kit are in a sealed container.

30. The kit according to claim 26, wherein said support and said plurality of scent members are modular components.
